(19) United States
(12) Patent Application Publication
(10) Pub. No.: US 2015/0367098 A1
(43) Pub. Date: Dec. 24, 2015

(54) CATHETER OR SHEATH ASSEMBLY

(71) Applicant: Cook Medical Technologies, LLC, Bloomington, IN (US)

(72) Inventors: Steen Aggerholm, St. Heddinge (DK); David Drewes, Bloomington, IN (US); Thomas A. Osborne, Bloomington, IN (US); Brett Baker, Ellettsville, IN (US)

(73) Assignee: COOK MEDICAL TECHNOLOGIES, LLC, Bloomington, IN (US)

(21) Appl. No.: 14/725,093

(22) Filed: May 29, 2015

(30) Foreign Application Priority Data
Jun. 23, 2014 (GB) 1411104.1

(51) Int. Cl. A61M 25/00

(52) U.S. Cl. CPC A61M 25/005; A61M 25/0012; A61M 2025/0059

(57) ABSTRACT

A catheter or sheath includes an inner layer made of a relatively hard material, an intermediate layer formed of a strengthening member and being provided with interstitial material in the spaces between the elements of the strengthening member. Overlying the intermediate layer is an outer layer of, for example, polyamide. The interstitial material is relatively soft, which allows the catheter to be bent to smaller radii of curvature than prior art catheters with reduced risk of kinking.

### RELATED APPLICATIONS

The present patent document claims the benefit of priority to Great Britain Patent Application No. GB1411104.1, filed Jun. 23, 2014, and entitled “CATHETER OR SHEATH ASSEMBLY,” the entire contents of each of which are incorporated herein by reference.

### BACKGROUND

1. Technical Field

The present invention relates to a catheter or sheath for an introducer assembly.

2. Background Information

Catheters and sheaths (hereinafter referred to generally as sheaths) are in common use in medical procedures. These may be relatively short but in many instances can be of significant length. This is the case for endoluminal procedures, in which the sheath is introduced into a patient from a percutaneous entry point and then fed through the patient's vasculature to the location of treatment. Such sheaths must be flexible enough to be able to curve and bend through a patient's vessels, often while being trained over a guide wire. It is important in this regard that the sheath is flexible enough not to damage the vessel walls during its passage therethrough, that is, to have good trackability. However, the sheath must also have good strength to ensure its internal lumen does not constrict in use. Any such deformation could adversely affect the state or deployment of any medical device or tool disposed in the sheath.

Moreover, the sheath must also have good kink resistance. Any kinking of the sheath will generally render it useless and result in an abortive medical procedure. Problems of kinking can occur not only within the patient's vasculature, particularly through tight curves, but also outside the patient, where the clinician will typically apply significant pushing and twisting forces at the proximal end of the sheath.

In addition to the above, it is optimal to minimize the thickness of the walls of the sheath as this improves trackability and also minimizes the footprint, that is, the outer diameter of the assembly. However, thin walled sheaths tend to be weaker and have higher risk of kinking.

Examples of prior art catheters and sheaths can be found in U.S. Pat. No. 5,669,920, U.S. Pat. No. 7,674,421, US 2006/0151923, US 2010/0217257, US 2011/0282288 and EP 0,956,878.

### BRIEF SUMMARY

The present invention seeks to provide an improved catheter or sheath particularly for an introducer assembly and more generally for medical applications.

According to an aspect of the present invention, there is provided a catheter or sheath comprising an inner layer of tubular form having a first compressibility, the inner layer having a length and an outer periphery; a strengthening member overlying the inner layer, the strengthening member being formed of one or more elongate elements extending around the periphery and along the length of the inner layer, there being interstices between the elongate element or elements, the strengthening member having an inner side facing the outer periphery of the inner layer and an outer side facing outwardly of the inner layer, and a thickness between the inner and outer sides; an interstitial material disposed in the interstices between the strengthening element or elements of the strengthening member and extending through the thickness of the strengthening member, the interstitial material having a compressibility greater than the first compressibility; and an outer layer overlying the strengthening member and interstitial material.

The inner layer of the catheter or sheath provides a stable inner wall for supporting the shape of the lumen of the catheter or sheath and in the preferred embodiment for providing a smooth surface for ease of sliding components through the catheter or sheath. The more compressible interstitial material enables the strengthening elements of the strengthening member to close towards one another when the catheter or sheath is curved, enabling it to attain tighter radii of curvature than conventional sheaths. The outer layer is able to provide a smooth outer surface to the catheter or sheath and also, in the preferred embodiments, an outer layer which is relatively hard, which can increase resistance to kinking. In this regard, the outer layer preferably has a compressibility less than the compressibility of the interstitial material, or a hardness greater than a hardness of the interstitial material.

Advantageously, the inner layer has a hardness greater than a hardness of the interstitial material.

The inner layer may have a hardness of around 40 to 60 on the Shore D scale; the outer layer may have a hardness of around 40 to 60 on the Shore D scale; while the interstitial material may have a hardness of around 30 on the Shore D scale.

In the preferred embodiment, the outer periphery of the inner layer is substantially smooth and the strengthening member abuts the inner layer, in particular it lies on the inner layer without being pressed or partially embedded thereinto.

Advantageously, the outer layer lies on the strengthening element without extending into the thickness of the strengthening member.

Advantageously, the interstitial material is located solely in the interstices between the strengthening element or elements. In other words, the strengthening element is not embedded in the interstitial material but is free of interstitial material at its upper and lower surfaces, that is, the surfaces thereof which touch or abut the inner and outer layers. This reduces the thickness of the catheter or sheath and also increases its kink resistance by minimizing the amount of material having greater compressibility.

In one embodiment, the outer layer is in contact with the strengthening member and the interstitial material. In other embodiments, the strengthening member is fully embedded in the interstitial material.

In an embodiment, the interstitial material has a softening or melting temperature less than a softening or melting temperature of the inner layer. This characteristic can be useful in the manufacture of the catheter or sheath.

In a preferred embodiment, the interstitial material is formed of randomly oriented polymer material, while the inner layer is made of longitudinally oriented polymer material.

In a practical embodiment the inner layer may be made of polytetrafluoroethylene such as Teflon, the interstitial material is made of functionalized polyolefin resin or polyurethane resin and the outer layer is made of polyamide such as Nylon.

According to another aspect of the present invention, there is provided a method of manufacturing a catheter or sheath comprising the steps of: forming an inner layer of tubular shape having a first compressibility, the inner layer having a length and an outer periphery; disposing a strengthening member over the inner layer, the strengthening member being formed of one or more elongate elements extending around the periphery and along the length of the inner layer, there being interstices between the elongate element or elements, the strengthening member having an inner side facing the outer periphery of the inner layer and an outer side facing outwardly of the inner layer, and a thickness between the inner and outer sides; disposing an interstitial material disposed in the interstices between the strengthening element or elements of the strengthening member so that the interstitial material extends through the thickness of the strengthening member, the interstitial material having a compressibility greater than the first compressibility; and forming an outer layer over the strengthening member and interstitial material.

The inner layer may be formed by extrusion and the interstitial layer by melting onto the inner layer and superposed strengthening member.

The outer layer may be formed by heat shrinking a cover over the outer layer, thereby to press the outer layer onto the strengthening member and interstitial material, then removing the cover.

### BRIEF DESCRIPTION OF THE DRAWINGS

Embodiments of the present invention are described below, by way of example only, with reference to the accompanying drawings, in which:

FIG. 1 is a schematic diagram in longitudinal cross-section of a preferred embodiment of catheter or sheath structure; and

FIG. 2 is a schematic diagram depicting a preferred method of manufacturing a catheter or sheath according to the embodiment of FIG. 1.

### DETAILED DESCRIPTION OF THE DRAWINGS AND THE PRESENTLY PREFERRED EMBODIMENTS

The drawings show embodiments of a catheter or sheath and of a manufacturing process in schematic form only and not to scale. The skilled person will appreciate that the layers of the sheath will have thicknesses dependent upon the material and components used, as well as the relative dimensions of the sheath.

The teachings herein are applicable to any catheter or sheath used in medical procedures, particularly for passage through a patient's vasculature. The teachings herein are also particularly suited for the outer sheath of an introducer assembly. In the description which follows catheters and sheaths will be collectively referred to as a sheath.

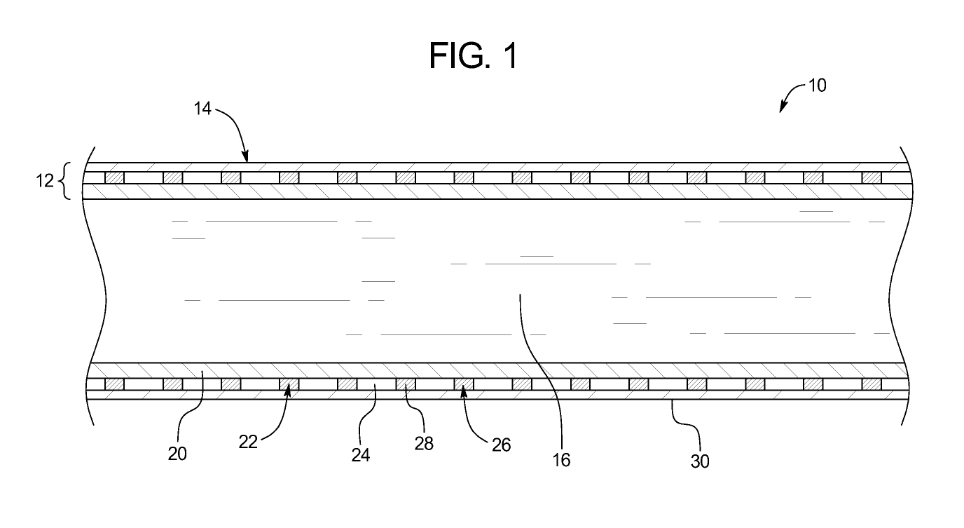

Referring first to FIG. 1, there is shown in schematic form and in longitudinal cross-section a preferred embodiment of sheath 10. The sheath 10 has a wall 12 formed of a plurality of layers 14 described below, and an internal lumen 16 through which medical devices and medical tools, for example, can be made to pass. The sheath 10 may constructed in a variety of diameters from several tenths of a French to one or just a few French (a French being 0.3 mm). It may even be constructed to have a much larger diameter.

The sheath 10 typically has a round internal lumen 16 and its wall 12 is typically annular in transverse cross-section with a uniform wall thickness in all radial orientations and along its length. It is not excluded, however, that the wall 12 could have varying thicknesses over the longitudinal extent of the sheath 10.

The wall 12 of the sheath 10 includes a first or inner layer 20 made of a relatively hard and low friction material, for instance a polymer material. An example of a suitable material is polytetrafluoroethylene (PTFE) such as Teflon™.

In the preferred embodiment, the material of the inner layer 20 is formed of longitudinally oriented polymer chains, that is, oriented in the longitudinal direction of the sheath 10. Such a structure reduces the compressibility of the material in the longitudinal direction and thereby provides longitudinal stability to the sheath 10. This enhances the pushability of the sheath 10, that is, its ability to be pushed through a patient's vasculature.

The inner layer 20 is preferably a unitary layer with the same material throughout its thickness. It is not excluded, though, that it may have one or more sublayers or one or more coatings.

A second or intermediate layer 22 overlies the inner layer 20 and includes a strengthening member 24 disposed, in the preferred embodiment, in direct contact with, that is in abutment with, the outer surface of the inner layer 20. The strengthening member 24 may be a coil or braid. In the case of a coil, this is preferably of a thin strip of material such that the width of the coil is several times its thickness. In the case of a braid, this is typically made of one or more lengths of wire braided together. Typically, the strengthening member 24 is made of a spring metal or metal alloy of which examples are known in the art.

The strengthening member 24 provides between the elements of strengthening material, that is, between turns of coil or between braids, interstitial spaces 26. Interstitial material 28 fills the interstitial spaces 26, as can be seen in FIG. 1. The interstitial material 28 has a greater compressibility than the compressibility of the material of the inner layer 20, particularly in the longitudinal direction of the sheath, in other words in the direction of greatest compressive force experienced during curving of the sheath 10, as explained below. The interstitial material 28 could be described as being relatively soft compared the inner layer 20 and a third or outer layer 30 described below.

The interstitial material 28 may be or include a polymer material. An illustrative embodiment uses a functionalized polyolefin resin or polyurethane resin, for example Admer, such as Admer SF755A, produced by Mitsui. These are preferred materials but other materials may be used. Another example is a relatively soft polyether block amide such as Pebax™.

It is preferred that the interstitial material 28 is formed of randomly oriented polymer chains, which enhances the material's compressibility without reducing its strength.

Advantageously, the interstitial material 28 has a lower softening or melting temperature than the softening or melting temperature of the material of the inner layer 20. This enables the interstitial material 28 to be applied to the inner layer 20 in a softened or melted state without risking damage to the structure of the inner layer 20. In practice, such interstitial material 28 can be made to flow into the interstitial spaces 26, thereby to fill these completely and also to bond to the inner layer 20. Bonding of the interstitial material 28 to the inner layer 20 may thus be by its being softened or melted during its application to the inner layer 20, but may also be achieved by means of a bonding agent. An embodiment of manufacturing method is described below.

In the preferred embodiment, the interstitial material 28 only fills the interstitial spaces 26, that is, it does not envelop the strengthening member 24. In other words, the interstitial material has a thickness no greater than the thickness of the strengthening member 24 and does not extend beyond the inner and outer surfaces sides of the strengthening member 24. Thus, as can be seen in FIG. 1, the interstitial material 28 lies alongside the elements of the strengthening member 24 such that both the interstitial material 28 and the strengthening member 24 are in direct abutment or contact with the inner layer 20, as well as of the outer layer 30 described below. This arrangement provides a particularly compact structure with minimal wall thickness and thinner than prior art structures. As is explained below, the interstitial material could have a thickness the same as the thickness of the strengthening member 24 or could be thicker than the strengthening member 24, in which case it will at least partially embed the strengthening member 24 therewithin.

It has also been found that this arrangement provides a sheath structure which is more resistant to kinking, particularly in cases where the interstitial material 28 is significantly softer than the material of the inner layer 20.

It is not excluded that in some embodiments the interstitial material 28 may overlie the inner and/or outer surfaces of the strengthening member 24, but any such overlap should be as thin as possible. In other words, the strengthening member 24 may be completely embedded in the interstitial material, is which case the strengthening member 24 is not in direct contact with the inner layer 20 or the outer layer 30.

Overlying the second or intermediate layer 22 is a third or, in this embodiment, outer layer 30 which envelops the intermediate layer 22. The third layer 30 is preferably made of a material of lower compressibility than that of the interstitial material 28. The third layer 30 may, for example, be formed of polyamide such as Nylon, of polyether block amide such as Pebax™ or similar material.

In the preferred embodiment, the third layer 30 contacts directly the interstitial material as well as the upper or outer surface of the strengthening member 24. In practice, the third layer 30 is bonded at least to the interstitial material 28.

The third or outer layer 30 provides a smooth outer surface to the sheath 10, in particular as this is curved or bent, whereupon the differences in compressibility of the strengthening member 24 and interstitial material 28 would otherwise create edges in the outer surface of the sheath 10 were the outer layer 30 to be omitted. Furthermore, the outer layer 30 is able to provide a harder and more slippery outer surface to the sheath 10, which optimizes its trackability in a patient's vasculature and reduces the risk of damage to the vessel walls.

In the preferred embodiment, the inner layer 20 and outer layer 30 have a flexural modulus of 80 MPa or greater, while the interstitial material 28 has a flexural modulus of less than 80 MPa.

The inner layer 20 may have a durometer of around 40 to 60 on the Shore D scale, the interstitial material 28 a durometer of around 30 on the Shore D scale and the third layer 30 a durometer of around 40 to 60 on the Shore D scale.

A sheath 10 constructed as taught herein can have a relatively thin sheath wall 12 and improved bending characteristics, thus improved trackability in a patient's vasculature. This is particularly the case due to the provision of the soft interstitial material 28 in the gaps of the strengthening member 24. This material 28 compresses to allow the turns of the coil or wire of the braid to move towards one another in the inside of the curve as the sheath 10 is bent. The structure enables the sheath 10 to be bent to smaller radii of curvature compared to known sheaths. Yet, the provision of the inner and outer layers 22, 30, in addition to the compressibility of the interstitial material 28, provide strength to the sheath 10, optimizing its kink resistance. This is in part due to the fact that the soft interstitial material 28 is confined substantially to the interstitial spaces 26, allowing direct transfer of bending forces through the thickness of the sheath wall 12 and in particular to the stronger or harder components of the structure. The preferred structure of sheath 10 can thus have greater resistance to kinking compared to conventional sheaths.

Furthermore, the structure can provide a sheath of reduced wall thickness 12 compared to prior art sheaths, which improves trackability and the ability to pass through smaller vessels.

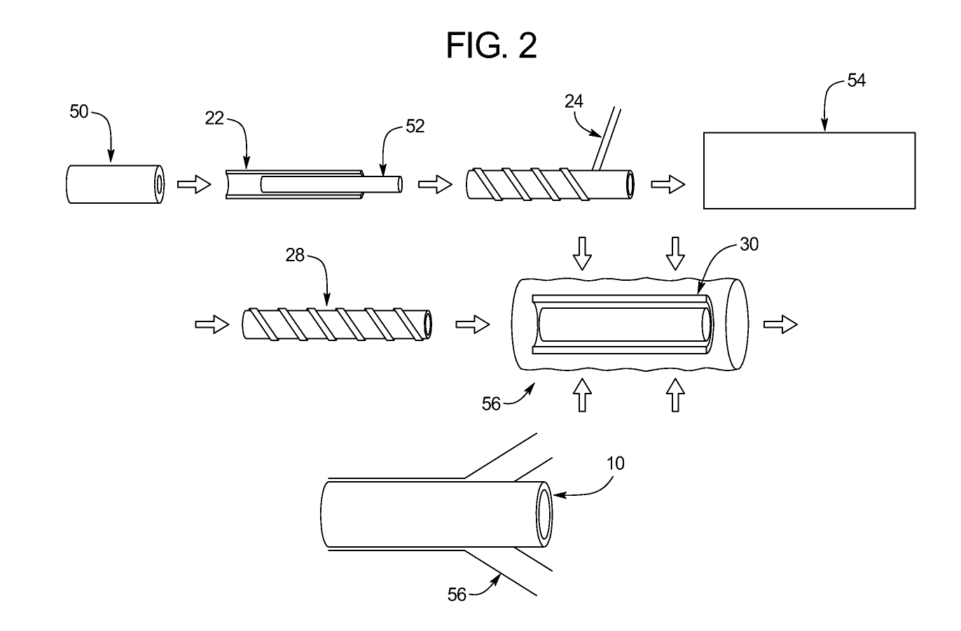

FIG. 2 shows an embodiment of assembly for the manufacture of a sheath 10 of the type disclosed herein.

The inner sheath layer 20 is preferably formed by extrusion through an extruder 50, which longitudinally orients the polymer chains forming the inner layer 20. This orientation of the polymer chains has the effect of reducing the longitudinal compressibility of the layer 20, as explained above. Given that the radial strength is provided by the strengthening member 24, the inner layer 20 does not have to exhibit particularly high radial strength of its own, which enables the thickness of the inner layer 20 to be reduced.

Once extruded, the inner layer 20 is cut to the desired length and then fitted onto a mandrel 52. The mandrel 52 has a diameter which is preferably a close match to the inner diameter of the inner layer 20 to ensure a tight fit of the tubular layer 20 thereon.

The outer surface of the inner layer may be treated to enhance its bonding properties with subsequent layers, in particular the interstitial material. Suitable treatment for an inner layer of PTFE can be with sodium solution, in which the sodium ions strip the fluorine from the top surface of the inner layer and replaces this with carbon, which will bond better to the interstitial material.

Once the inner layer 20 is positioned on the mandrel 52, the strengthening member 24 is placed onto the outside of the inner layer 20, for example by winding of a strengthening coil, as shown in the drawing. In the case where the strengthening member 24 is a braid, this is fitted over the inner layer 20 in conventional manner.

After this step, the structure is fed into a mold 54, whereupon the interstitial material 28 is applied to the structure and in particular into the interstitial spaces 24. This can be achieved by heat molding, which will cause the interstitial material to melt or soften before being pressed into the interstitial spaces, typically by conventional mold elements. After application, the assembly of inner layer 20, strengthening member 24 and interstitial material 28 is allowed to cool so as to harden and set the interstitial material 28 in position.

Next, a tube of third layer material 30 is located over the structure and, in this embodiment, a heat shrink wrap 56 applied around the outside. The heat shrink wrap 56 presses the third layer 30 against the intermediate layer 22. Application of heat at this stage ensures the third layer 30 softens sufficiently to be moldable and to bond to the intermediate layer 22.

It will be appreciated that bonding of the various layers, in particular the polymer elements, may be by heat or fusion bonding, but in other embodiments one or more of the layers or elements may be bonded by means of a bonding agent. It is preferred, though, to avoid use of separate layers of bonding agent.

It is envisaged that in some embodiments the interstitial material may form a layer having significant thickness, particularly being 50 to 60 percent of the overall thickness of the wall of the sheath. In such embodiments, the strengthening member would be entirely embedded in the interstitial material.

In other embodiments, the interstitial material may extend over either of the inner or outer surfaces of the strengthening member, so as to be disposed between the strengthening members and the respective one of the inner and outer layers of the sheath.

It is generally preferred that the sheath have a circular transverse cross-section so as to have uniform bendability and trackability in all radial directions, as is conventional in the art. It is not excluded, though, that it may have a non-round transverse cross-section, for instance oval. A non-round cross-section will cause the sheath to have different bending properties in different angular directions, which can be useful in the deployment of oriented medical devices.

It is to be understood that the above described embodiments are not limiting of the more general teachings and invention disclosed herein and the skilled person will recognize various modifications and additions which are intended to fall within the scope of the claims which follow.

1. A catheter or sheath comprising:
an inner layer of tubular form having a first compressibility, the inner layer having a length and an outer periphery;
a strengthening member overlying the inner layer, the strengthening member being formed of one or more elongate elements extending around the periphery and along the length of the inner layer, there being interstices between the elongate element or elements, the strengthening member having an inner side facing the outer periphery of the inner layer and an outer side facing outwardly of the inner layer, and a thickness between the inner and outer sides;
an interstitial material disposed in the interstices between the strengthening element or elements of the strengthening member and extending through the thickness of the strengthening member, the interstitial material having a compressibility greater than the first compressibility; and
an outer layer overlying the strengthening member and interstitial material.

2. A catheter or sheath according to claim 1, wherein the outer layer has a compressibility less than the compressibility of the interstitial material.

3. A catheter or sheath according to claim 1, wherein the outer layer has a hardness greater than a hardness of the interstitial material.

4. A catheter or sheath according to claim 1, wherein the inner layer has a hardness greater than a hardness of the interstitial material.

5. A catheter or sheath according to claim 1, wherein the inner layer has a hardness of around 40 to 60 on the Shore D scale.

6. A catheter or sheath according to claim 1, wherein the outer layer has a hardness of around 40 to 60 on the Shore D scale.

7. A catheter or sheath according to claim 1, wherein the interstitial material has a hardness of around 30 on the Shore D scale.

8. A catheter or sheath according to claim 1, wherein the outer periphery of the inner layer is substantially smooth.

9. A catheter or sheath according to claim 1, wherein the strengthening member abuts the inner layer.

10. A catheter or sheath according to claim 9, wherein the strengthening element lies on the inner layer without being embedded thereinto.

11. A catheter or sheath according to claim 1, wherein the outer layer lies on the strengthening element without extending into the thickness of the strengthening member.

12. A catheter or sheath according to claim 1, wherein the interstitial material is located solely in the interstices between the strengthening element or elements.

13. A catheter or sheath according to claim 1, wherein the interstitial material has a thickness no greater than the thickness of the strengthening member.

14. A catheter or sheath according to claim 1, wherein the outer layer is in contact with the strengthening member and the interstitial material.

15. A catheter or sheath according to claim 1, wherein the strengthening member is fully embedded in the interstitial material.

16. A catheter or sheath according to claim 1, wherein the interstitial material has a softening or melting temperature less than a softening or melting temperature of the inner layer.

17. A catheter or sheath according to claim 1, wherein the interstitial material is formed of randomly oriented polymer material.

18. A catheter or sheath according to claim 1, wherein the inner layer is made of longitudinally oriented polymer material.

19. A catheter or sheath according to claim 1, wherein the inner layer is made of polytetrafluoroethylene.

20. A catheter or sheath according to claim 1, wherein the interstitial material is made of functionalized polyolefin resin or polyurethane resin.

21. A catheter or sheath according to claim 1, wherein the outer layer is made of polyamide.

22. A method of manufacturing a catheter or sheath comprising the steps of:
forming an inner layer of tubular shape having a first compressibility, the inner layer having a length and an outer periphery;
disposing a strengthening member over the inner layer, the strengthening member being formed of one or more elongate elements extending around the periphery and along the length of the inner layer, there being interstices between the elongate element or elements, the strengthening member having an inner side facing the outer periphery of the inner layer and an outer side facing outwardly of the inner layer, and a thickness between the inner and outer sides;
disposing an interstitial material disposed in the interstices between the strengthening element or elements of the strengthening member so that the interstitial material extends through the thickness of the strengthening member, the interstitial material having a compressibility greater than the first compressibility; and
forming an outer layer over the strengthening member and interstitial material.

23. A method according to claim 22, wherein the inner layer is formed by extrusion and the interstitial material layer by melting onto the inner layer and superposed strengthening member.

24. A method according to claim 22, wherein the outer layer is formed by heat shrinking a cover over the outer layer, thereby to press the outer layer onto the strengthening member and interstitial material, then removing the cover.
